(19) United States
(12) Patent Application Publication
(10) Pub. No.: US 2025/0101361 A1
(43) Pub. Date: Mar. 27, 2025

(54) CELL OBSERVATION SYSTEM

(71) Applicants: CANON KABUSHIKI KAISHA, Tokyo (JP); CANON MEDICAL SYSTEMS CORPORATION, Tochigi (JP)

(72) Inventors: HISAFUMI EBISAWA, Tokyo (JP); YOJI YAMAMOTO, Tokyo (JP); ICHIRO HARADA, Kanagawa (JP)

(21) Appl. No.: 18/888,608

(22) Filed: Sep. 18, 2024

(30) Foreign Application Priority Data
Sep. 27, 2023 (JP) 2023-166289

(51) Int. Cl. C12M 1/34; C12M 1/00

(52) U.S. Cl. CPC C12M 41/36; C12M 23/22; C12M 31/02; C12M 41/06

(57) ABSTRACT

A cell observation system has: a part for a culturing container to be placed, the culturing container having a surface where cells are culturable and an optically transparent side face forming a peripheral wall of the surface; a light emission unit configured to irradiate the surface with irradiation light from the side face; and an image capturing unit configured to capture images of the surface and has at least any of features (a) controlling variation in an amount of light of the irradiation light, the surface being irradiated with the irradiation light, and the variation being due to absorption of light by a medium in the culturing container and (b) correcting an effect caused by variation in an amount of light of the irradiation light, the surface being irradiated with the irradiation light, and the variation being due to absorption of light by a medium in the culturing container.

### BACKGROUND OF THE INVENTION

### Field of the Invention

The present disclosure relates to a cell observation system.

### Description of the Related Art

In culturing cells, technologies to observe a wide culturing surface in a short time are important for cell culturing. For example, a ratio of cells occupying a culturing surface (confluency) can be used as an index of a passage timing or the like. Further, when a colony of clustered cells has been formed, it is also possible to observe the shape of the colony of cultured cells based on scattering of light irradiated. Japanese Patent Application Laid-Open No. 2016-077226 proposes a system that can observe colony shapes by irradiating a culturing container with light from the side face thereof and observing scattered light from the colony by using a camera installed under the culturing container.

### SUMMARY OF THE INVENTION

In the observation system that irradiates a culturing container with light from the side face thereof, however, there is a problem of occurrence of unevenness in an image or temporal variation in images of captured images.

The present disclosure has been made in view of the above problem.

The present inventors have considered that the following reason would cause unevenness in an image or temporal variation in images of captured images to occur in the observation system that irradiates a culturing container with light from the side face thereof. That is, in the observation system that irradiates a culturing container with light from the side face thereof, the irradiated light passes through in the culturing container and travels while being scattered by cells or the like near the culturing surface. Thus, in the culturing container, the amount of light more decreases on the side far from the light source than on the side close to the light source. Further, since culture media generally contain a pH indicator represented by phenol red and exhibit a color change in accordance with pH, variation in the amount of light occurs also due to such a color change in accordance with pH of a culture medium. Such variation in the amount of light of the irradiation light with which the culturing surface in the culturing container is irradiated as described above is considered to cause unevenness in an image or temporal variation in images of captured images.

Accordingly, it has been found that the above problem can be solved by providing a cell observation system that suppresses or corrects unevenness in an image or temporal variation in images of captured images due to in-plane or temporal variation in the amount of light of the irradiation light with which the culturing surface is irradiated. Furthermore, it has been found that the characteristics of a medium can be calculated and presented to the user based on unevenness in an image or temporal variation in images of captured images.

That is, the present disclosure provides a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face; and
an image capturing unit configured to capture an image of the culturing surface,
wherein the cell observation system has at least any one of features of
(a) controlling variation in an amount of light of the irradiation light, the culturing surface being irradiated with the irradiation light, and the variation being due to absorption of light by a medium in the culturing container, and
(b) correcting an effect caused by variation in an amount of light of the irradiation light, the culturing surface being irradiated with the irradiation light, and the variation being due to absorption of light by a medium in the culturing container.

Further, the present disclosure provides a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face;
an image capturing unit configured to capture an image of the culturing surface; and
an information processing unit,
wherein the information processing unit performs
an image information acquisition step of acquiring image information on a captured image from the image capturing unit,
a characteristic calculation step of calculating a characteristic of a medium in the culturing container from the image information, and
a characteristic output step of outputting the characteristic of the medium in the culturing container.

Further features of the present invention will become apparent from the following description of exemplary embodiments with reference to the attached drawings.

### BRIEF DESCRIPTION OF THE DRAWINGS

FIG. 1 is a schematic diagram illustrating a first embodiment.

FIG. 2 illustrates the absorption spectrum of phenol red.

FIG. 3 illustrates a preferred example of the spectrum of irradiation light.

FIG. 4 illustrates a preferred example of the spectrum of irradiation light.

FIG. 5 is a schematic diagram illustrating a second embodiment.

FIG. 6 is a schematic diagram illustrating steps performed by an information processing unit in the second embodiment.

FIG. 7 is a schematic diagram illustrating an example of a hardware configuration of the information processing unit.

FIG. 8 is a schematic diagram illustrating steps performed by the information processing unit in a third embodiment.

### DESCRIPTION OF THE EMBODIMENTS

The present disclosure provides a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face; and
an image capturing unit configured to capture an image of the culturing surface,
wherein the cell observation system has at least any one of features of
(a) controlling variation in an amount of light of the irradiation light, the culturing surface being irradiated with the irradiation light, and the variation being due to absorption of light by a medium in the culturing container, and
(b) correcting an effect caused by variation in an amount of light of the irradiation light, the culturing surface being irradiated with the irradiation light, and the variation being due to absorption of light by a medium in the culturing container.

Preferred embodiments of the present disclosure will be described below with reference to the drawings.

### First Embodiment

A first embodiment of the present disclosure is a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face; and
an image capturing unit configured to capture an image of the culturing surface,
wherein the cell observation system has at least any one of features of
(a) controlling variation in an amount of light of the irradiation light, the culturing surface being irradiated with the irradiation light, and the variation being due to absorption of light by a medium in the culturing container, and
(b) correcting an effect caused by variation in an amount of light of the irradiation light, the culturing surface being irradiated with the irradiation light, and the variation being due to absorption of light by a medium in the culturing container,
wherein the cell observation system has the feature of (a), and
wherein intensities of the irradiation light at a wavelength λ_{2A} [nm] and a wavelength λ_{2B} [nm] in a spectroscopic spectrum of the irradiation light are less than or equal to 50% of the intensity at a wavelength λ₁ [nm].

Note that the intensity is the maximum at the wavelength λ₁ [nm] in the spectroscopic spectrum of the irradiation light,
absorption is the maximum at the wavelength λ_{2A} [nm] in an absorption spectrum of the medium when the medium is acidic, and
absorption is the maximum at the wavelength λ_{2B} [nm] in an absorption spectrum of the medium when the medium is alkaline.

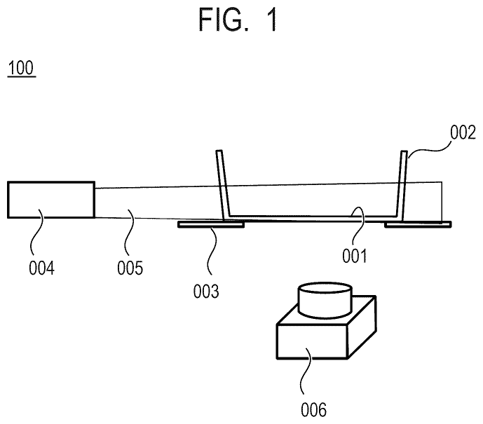

The present embodiment will be described below with reference to FIG. 1 to FIG. 5.

### System Configuration

As illustrated in the schematic diagram of FIG. 1, a cell culturing system 100 according to the present embodiment has a placement part 003, a light emission unit 004, and an image capturing unit 006. A culturing container 002 is placed on the placement part 003. The culturing container 002 is a holding container that holds a culture medium used for culturing cells, and a culturing surface 001 where cells can adhere and proliferate is provided at the bottom of the container. In FIG. 1, an opening corresponding to an image capturing region is provided in the placement part 003 so that the image capturing unit 006 arranged below the placement part 003 can capture an image of cells in the culturing container 002 via the transparent bottom face of the culturing container 002. The light emission unit 004 emits irradiation light 005 in the side face direction of the culturing container 002 to irradiate the culturing surface 001 or a part near the culturing surface 001 through the side face. The image capturing unit 006 captures an image of scattered light scattered from the irradiation light 005 with which cells that have adhered to the culturing surface 001 are irradiated and thereby acquires the shape of a colony or the like. Although FIG. 1 illustrates the example in which the image capturing unit 006 captures an image from below the culturing container 002, the image capturing unit 006 is not necessarily required to be arranged below the culturing container 002 and may be arranged above, diagonally, or the like.

### Culturing Container

As described above, the culturing container 002 holds a culture medium used for culturing cells and also holds cells arranged in the culture medium. Note that, while the cell observation system of the present disclosure does not include the culturing container 002 as a requirement, the cell observation system of the present disclosure can use the culturing container 002 described below.

Preferred materials of the culturing container 002 may be transparent resin, glass, or the like that enable easy machining and observation. In the case of resin, polystyrene, polycarbonate, acrylic, or the like can be used. Further, examples of the shape of the culturing container 002 may be those having a rectangular or circular bottom face. The culturing surface 001 is configured such that cells can adhere thereto and proliferate thereon and is preferably made of a transparent material that enables easy observation. In the culturing container 002, the culturing surface 001 may be the container bottom face or may be a component that is separate from the container and is arranged on the container bottom face. In terms of costs or production, however, it is preferable to configure the culturing surface 001 as a part of the culturing container 002 by using the same material as the culturing container 002.

In such a case, it is preferable to apply surface treatment such as plasma treatment so that cells are likely to adhere to only the culturing surface 001.

Any culturing container can be used as long as it is the culturing container as described above without limitation, and commercially available culturing dish, culturing flask, or the like can also be used.

### Placement Part

The placement part 003 is a table for installing the culturing container 002 thereon. The placement part 003 needs to be configured so that the image capturing unit 006 arranged below the placement part 003 can capture an image of cells in the culturing container 002 via the transparent bottom face of the culturing container 002. Thus, an opening corresponding to the image capturing region can be provided in the placement part 003. The major portion of the culturing surface 001 can be observed without being hidden by the placement part 003. It is desirable that the opening have a similar shape to the bottom face of the culturing container 002 and be slightly smaller than the bottom face so that the culturing container 002 can be stably placed and a range as wide as possible can be observed from the image capturing unit 006. For example, when the culturing container 002 is a circular Petri dish, the opening can be a circular opening slightly smaller than the Petri dish, and when the culturing container 002 is rectangular, the opening can be a rectangular opening slightly smaller than the culturing container 002.

Alternatively, it is also preferable to use a transparent member such as glass for the placement part 003 and, in such a case, it is possible to observe the culturing surface 001 without providing an opening.

The shape of the placement part 003 is preferably a shape not blocking the irradiation light 005 traveling to the culturing surface 001. For higher image quality of an acquired image, it is effective to provide a mask effect to block the irradiation light 005 emitted to outside of the culturing surface 001.

The placement part 003 may have a frame or a metal fitting for stably placing the culturing container 002.

### Light Emission Unit

The irradiation light 005 from the light emission unit 004 is preferably such light that is emitted to only the culturing surface 001 with stray light reduced as much as possible in terms of contrast of the image quality. Further, to prevent unevenness in a captured image plane from occurring, it is preferable that the amount of light of the irradiation light 005 with which the image capturing region is irradiated be even. From the above, as for the light emission unit 004, it is preferable to select a light source that can emit the irradiation light 005 that mainly irradiates the culturing surface 001 and has an even amount of light on the culturing surface. For example, it is preferable to use a line type LED light source having high directionality, a line light guide in which emission ends of the bundle fibers are arranged in a line, or the like. Such selection of a light source makes it possible to efficiently irradiate only the part at or near the culturing surface 001. The side face of the culturing container 002 is irradiated with the irradiation light 005 from the light emission unit 004. Thus, if the irradiation light 005 has a large spread angle, the irradiation light is diffused, and this causes a difference in intensity of the irradiation light 005 irradiated on the culturing surface 001 to occur between the side close to the light emission unit 004 and the side far from the light emission unit 004. To reduce this difference, it is preferable that the irradiation light 005 be nearly parallel light having a suppressed spread angle. It is thus effective to use a collimate member for the light emission unit 004. An example of the collimate member may be a cylindrical lens.

Further, to restrict the emission range of the light emission unit 004 to the culturing surface 001, it is effective to install a mask between the light emission unit 004 and the culturing surface 001, for example, on the side face of the culturing container 002. When a mask is installed, the light from the light emission unit 004 may expand in a wide range, it is no longer necessary to configure the light emission unit 004 into a line type, and choices of the light emission unit 004 will be expanded.

### Spectrum Characteristic of Irradiation Light

As the light emission unit 004, a light source of a halogen lamp, an LED lamp, or the like can be used. The irradiation light 005 emitted by the light emission unit 004 is required to have spectrum characteristics that cause less damage to cells. Furthermore, the irradiation light 005 preferably has spectrum characteristics that cause less absorption by the medium in the culturing container 002 in order to control variation in the amount of light of the irradiation light 005 due to absorption of light by the medium in the culturing container 002. When light having a wavelength corresponding to a low absorptivity due to the medium is selected as the irradiation light 005, the difference in the amount of light between the irradiation light 005 irradiated on the side close to the light emission unit 004 and that on the side far from the light emission unit 004 can be suppressed in the culturing container 002, and the variation in the amount of light of the irradiation light 005 due to absorption of light by the medium can be suppressed. Further, when the absorption characteristic of a medium changes in accordance with conditions such as pH or the like, it is preferable to emit light having a wavelength that is less likely to be affected by the change, and it is possible to suppress the change in the amount of irradiation light due to a change in conditions.

The cell culturing medium that is a general medium contains a pH indicator such as phenol red, and the absorption characteristic changes due to a change in pH.

It is thus preferable that the irradiation light have a low intensity at the wavelength λ_{2A} [nm] and the wavelength λ_{2B} [nm] in the spectroscopic spectrum thereof, and intensities at the wavelength λ_{2A} [nm] and the wavelength λ_{2B} [nm] are preferably less than or equal to 50% of the intensity at the wavelength λ₁ [nm], where the wavelength λ₁ [nm] is the wavelength at which the intensity is the maximum in the spectroscopic spectrum of the irradiation light, the wavelength λ_{2A} [nm] is the wavelength at which absorption is the maximum in the absorption spectrum of the medium when the medium is acidic, and the wavelength λ_{2B} [nm] is the wavelength at which absorption is the maximum in the absorption spectrum of the medium when the medium is alkaline.

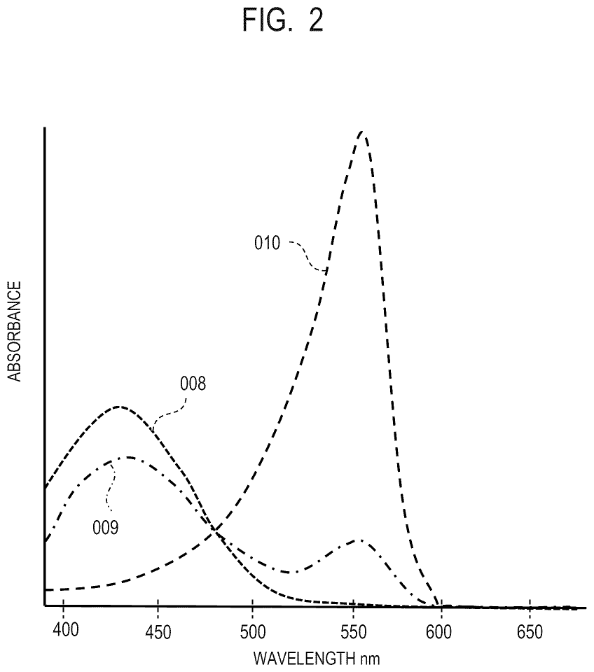

FIG. 2 represents absorption characteristics in accordance with pH of phenol red.

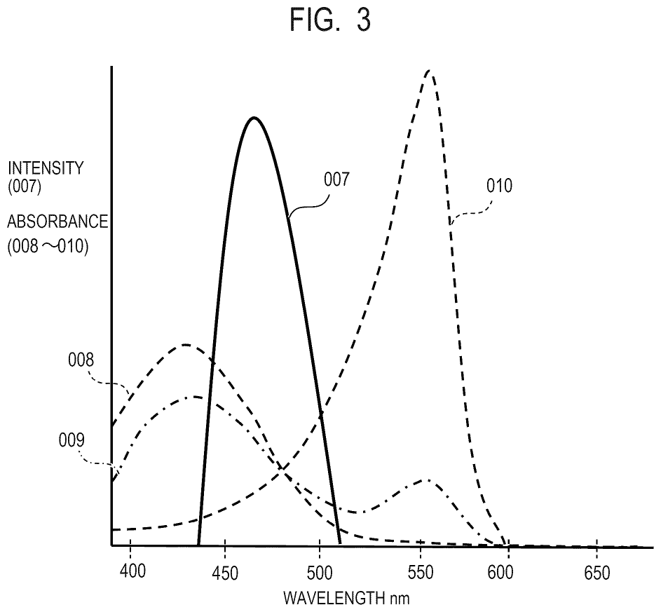

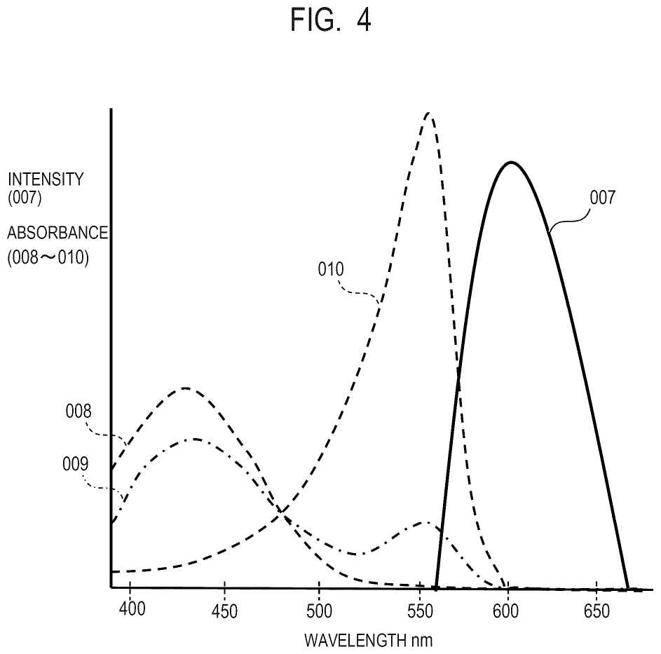

While a new culture medium is alkaline and has a red-violet color, the culture medium gradually changes to acidic by culturing and takes on a yellow tinge. In FIG. 2, the number 008 represents the absorption spectrum of acidic phenol red, the number 009 represents the absorption spectrum of neutral phenol red, and the number 010 represents the absorption spectrum of alkaline phenol red. As illustrated in FIG. 2, since phenol red has a minute absorptivity at a wavelength of 600 nm or higher regardless of pH, it is preferable to employ light having a wavelength of 600 nm as illustrated in FIG. 4 as the irradiation light 005. Further, when the phenol red has a relatively low absorptivity at a wavelength of 480 nm or therearound and has a constant isosbestic point regardless of pH even when pH varies. It is also effective to employ light having a wavelength near the isosbestic point of phenol red as illustrated in FIG. 3 as the irradiation light 005 because the variation in the intensity of the irradiation light 005 is less likely to be caused by a change in pH. Note that the number 007 in FIG. 3 represents the spectroscopic spectrum of the irradiation light.

Although a unimodal spectrum is illustrated as an example as the spectroscopic spectrum 007 of the irradiation light 005 in FIG. 3 and FIG. 4, the spectroscopic spectrum may be bimodal or multimodal, and the irradiation light 005 may have a maximum intensity at a wavelength of 480 nm or therearound and at a wavelength of 600 nm or higher, for example. As for recommended spectrum characteristics, the peak wavelength at which the intensity exhibits the maximum value is preferably within a range of 450 nm to 500 nm or a range of 600 nm or higher. Further, it is more preferable that the intensities at a wavelength of 430 nm and 560 nm be less than or equal to 50% of the maximum value.

When the medium contains a pH indicator other than phenol red, it is preferable to employ the irradiation light 005 having spectrum characteristics corresponding to the range described above in accordance with the characteristics of each pH indicator.

Known pH indicators may be applied to the pH indicator contained in the medium without limitation, and the pH indicator contained in the medium may be, as examples, phenol red, methyl violet, brilliant green, metanyl yellow, 4-phenylazodiphenylamine, metacresol purple, thymol blue, 2,8-dinitrophenol, 2,4-dinitrophenol, methyl yellow, ethyl orange, bromophenol blue, Congo red, methyl orange, naphthyl red, alizarin red, bromocresol green, 2,5-dinitrophenol, methyl red, lachmoid, ethyl red, p-nitrophenol, bromocresol purple, bromophenol red, bromothymol blue, neutral red, rosolic acid, a-naphtholphthalein, cresol red, phenolphthalein, p-xylenol blue, a-cresolphthalein, p-naphtholbenzene, thymolphthalein, Alizarin Yellow G, Alizarin Yellow R, tetryl, Tropeolin O, indigo carmine, or the like. For the absorption characteristics of each indicator, the distributor's catalog or the like can be referred.

The effect of suppressing variation in the amount of light due to absorption of light by the medium achieved by using the irradiation light 005 as described above becomes more notable as the image capturing range becomes wider, that is, the difference in optical length in the image capturing region becomes larger. A more significant effect is resulted when a generally used dish having a width of 30 mm or the culturing container 002 having a culturing surface area greater than this dish is used. That is, when the image capturing unit captures an image of a region of 30 mm×30 mm or greater, the cell observation system of the present embodiment achieves a more notable effect.

### Image Capturing Unit

The image capturing unit 006 may be any unit as long as it can acquire an image of cells that have adhered to the culturing surface 001, and a so-called digital camera or the like with CMOS, CCD, and the like can be used.

### Second Embodiment

The second embodiment of the present disclosure is a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face; and
an image capturing unit configured to capture an image of the culturing surface,
wherein the cell observation system has at least any one of features of
(a) controlling variation in an amount of light of the irradiation light, the culturing surface being irradiated with the irradiation light, and the variation being due to absorption of light by a medium in the culturing container, and
(b) correcting an effect caused by variation in an amount of light of the irradiation light, the culturing surface being irradiated with the irradiation light, and the variation being due to absorption of light by a medium in the culturing container,
wherein the cell observation system has the feature of (b) and further has an information processing unit, and
wherein the information processing unit performs
an image information acquisition step of acquiring image information on an image captured by the image capturing unit,
an irradiation light variation information acquisition step of acquiring irradiation light variation information, and
an image processing step of generating a processed image based on the image information and the irradiation light variation information.

### Configuration of Cell Observation System

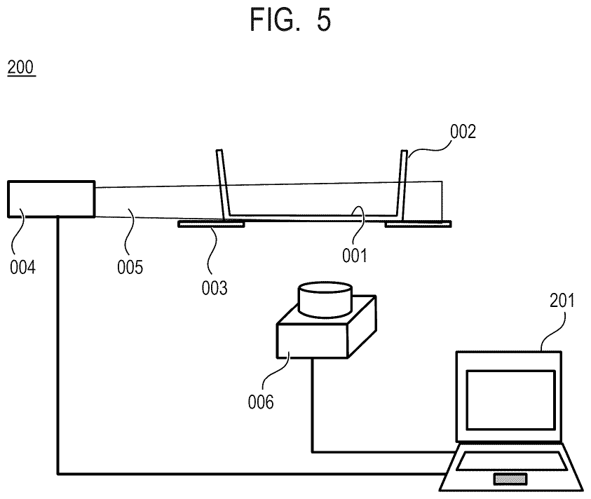

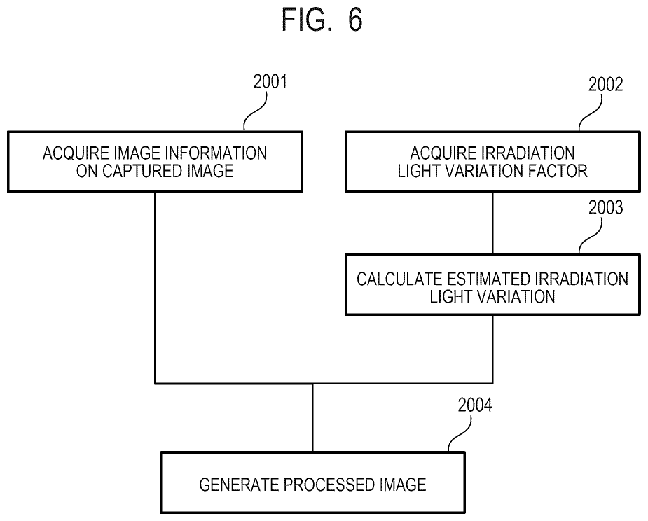

A cell observation system 200 of the present embodiment will be described with reference to FIG. 5 to FIG. 7. As illustrated in FIG. 5, the cell observation system of the present embodiment further has an information processing unit 201 in addition to the features of the first embodiment. As illustrated in FIG. 6, the information processing unit 201 acquires image information on images captured by the image capturing unit 006 (image information acquisition step 2001). Further, on the other hand, the information processing unit 201 acquires an irradiation light variation factor that causes variation in the intensity of the irradiation light 005 (irradiation light variation factor acquisition step 2002) and calculates an estimated irradiation light variation based on the acquired irradiation light variation factor (estimated irradiation light variation calculation step 2003). The information processing unit 201 then generates a processed image based on the image information and the estimated irradiation light variation (image processing step 2004). It is preferable for the information processing unit 201 to be connected to the image capturing unit 006 in order to transfer information to each other. Furthermore, the information processing unit 201 may be connected to the placement part 003, the light emission unit 004, or the like. Note that “connection” as used herein refers to a capability of transferring information regardless of whether the connection is wired or wireless.

### Information Processing Unit

The information processing unit 201 may have an image information acquisition unit for performing the image information acquisition step 2001, an irradiation light variation factor acquisition unit for performing the irradiation light variation factor acquisition step 2002, an estimated irradiation light variation calculation unit for performing the estimated irradiation light variation calculation step 2003, an image processing unit for performing the image processing step 2004, and a storage unit that stores an irradiation light variation factor or the like.

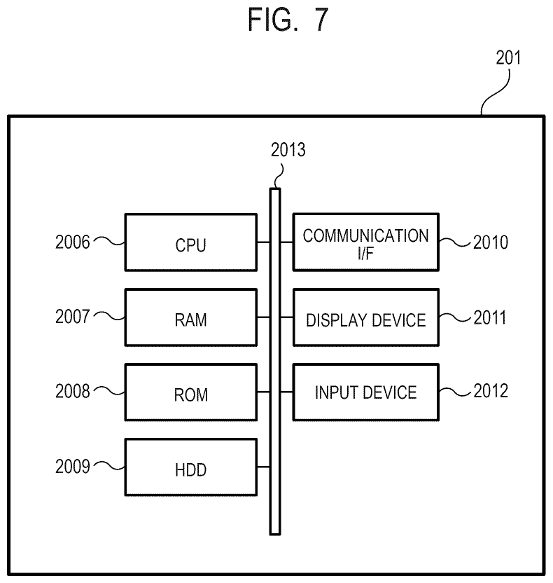

The information processing unit 201 has functions of a computer. FIG. 7 illustrates an overview of the hardware configuration of the information processing unit 201 as an example. The information processing unit 201 may be integrally configured with a desktop personal computer (PC), a laptop PC, a tablet PC, a smartphone, or the like. The information processing unit 201 includes a central processing unit (CPU) 2006, a random access memory (RAM) 2007, a read only memory (ROM) 2008, and a hard disk drive (HDD) 2009 in order to implement functions as a computer that performs computation and storage. Further, the information processing unit 201 includes a communication interface (I/F) 2010, a display device 2011, and an input device 2012. The CPU 2006, the RAM 2007, the ROM 2008, the HDD 2009, the communication I/F 2010, the display device 2011, and the input device 2012 are connected to each other via a bus 2013. Some of these functions may be formed of an external device. For example, the display device 2011 and the input device 2012 may be external devices separate from the portion configuring the functions of the computer including the CPU 2006 and the like. The CPU 2006 performs predetermined operations in accordance with a program stored in the RAM 2007, the HDD 2009, or the like and also has a function of controlling each unit of the information processing unit 201. The RAM 2007 is formed of a volatile storage medium and provides a temporarily memory region required for the operation of the CPU 2006. The ROM 2008 is formed of a nonvolatile storage medium and stores necessary information such as a program used for the operation of the information processing unit 201. The HDD 2009 is a storage device formed of a nonvolatile storage medium. The communication I/F 2010 is a communication interface based on the specification such as Wi-Fi (registered trademark), 4G, or the like, which is a module for communicating with other devices. The display device 2011 is a liquid crystal display, an organic light emitting diode (OLED) display, or the like and is used for displaying moving images, static images, texts, or the like. The input device 2012 is a button, a touch screen, a keyboard, a pointing device, or the like and is used by the user for operating the information processing unit 201. The display device 2011 and the input device 2012 may be integrally formed as a touch screen.

Note that the hardware configuration illustrated in FIG. 7 is an example, and a device other than the above may be added, or some of the devices may be omitted. Further, some of the devices may be replaced with another device having a similar function. Further, some of the functions may be provided by another device via a network, or the functions forming the present embodiment may be distributed and implemented in a plurality of devices. For example, the HDD 2009 may be replaced with a solid state drive (SSD) with a semiconductor device such as a flash memory or may be replaced with cloud storage. The CPU 2006 loads a program stored in the ROM 2008 or the like into the RAM 2007 to execute the program and thereby realizes the image information acquisition step 2001, the irradiation light variation factor acquisition step 2002, the estimated irradiation light variation calculation step 2003, and the image processing step 2004.

### Image Information Acquisition Step

The image information may be any information based on captured images and can use image data at a certain time point or a plurality of time points of the overall image in the culturing surface. The image information may include, for example, a distribution of colors, a change in color or concentration, the number or the density of cultured cells or colony, the size or the shape of a cell, information on an average in-plane output distribution, information on reflected light from the culturing container 002, or the like.

One or multiple pieces of image information can be acquired from one or multiple images. The multiple pieces of image information may be pieces of image information on a plurality of different regions within the culturing surface or may be pieces of image information at different time points for different regions or the same region. Based on the multiple pieces of image information, information calculated from the difference therebetween or the sum thereof may be used as the image information. Among other things, since comparison of these pieces of image information for different positions in the culturing surface 001 or comparison of the pieces of image information at different time points for the same position may be clues for inferring the change in color or the change in pH of the medium, it is useful to use multiple pieces of image information. When using multiple pieces of image information, use of image information on the closest region to the light emission unit 004 and the farthest region from the light emission unit 004 in the culturing surface 001 enables efficient acquisition of an irradiation light variation factor due to the difference in the distance from the light emission unit 004. Similarly, use of multiple pieces of image information that are temporally distant from each other enables efficient acquisition of an irradiation light variation factor due to a temporal change.

### Irradiation Light Variation Factor Acquisition Step

A factor of in-plane or temporal variation in the amount of light of the irradiation light 005 with which the culturing surface 001 is irradiated is referred to as an irradiation light variation factor.

The irradiation light variation factor may include, for example, information on irradiation light, information on a culturing container, and information on a medium or the like in a culturing container. The information on irradiation light may include the type of the light source of the light emission unit 004, the intensity, the spectroscopic spectrum, or the irradiation distribution of the irradiation light 005, or the like. The information on a culturing container may include the type, the material, the size, the capacity, the shape, the manufacturer, the model, or the like of the culturing container 002. The information on a medium or the like in a culturing container may include the type of pigments contained in the medium, the absorption characteristics of the medium or pigments contained in the medium, the liquid amount of the medium, the pH of the medium, the elapsed time from start of culturing, the type of cultured cells, the number of cultured cells, the state of proliferation of cultured cells, culturing conditions, or the like. The irradiation light variation factor is stored in the storage unit of the information processing unit 201, and this storing operation can be performed in accordance with user input or user selection from choices. Alternatively, the storing operation may be performed by automatic entry by the cell observation system. Examples of the automatic entry may be to determine the type of the culturing container 002 from image information acquired by the image capturing unit 006, install a weight sensor to the placement part 003, calculate the liquid amount of the medium, or the like to input these results to the storage unit. Information on correspondence between captured image information and irradiation light variation factors may be stored in the information processing unit 201 in advance, and an irradiation light variation factor may be calculated from this information. Alternatively, a reasoner may be provided, the reasoner may be used to infer an irradiation light variation factor from the captured image information, and the inferred result may be determined as the irradiation light variation factor. For example, the reasoner can use a learning model obtained by using training data in which image information is used as input data and irradiation light variation factors are used as output data.

### Estimated Irradiation Light Variation Calculation Step, Image Processing Step

Based on an acquired irradiation light variation factor, an estimated irradiation light variation is calculated as to how much the variation of the irradiation light 005 due to the irradiation light variation factor was in the culturing surface 001 to be captured. For example, it is here assumed that the temporal change in pH of the medium is acquired as the irradiation light variation factor. Based on the temporal change in color of the medium involved therein, information on the spectroscopic spectrum of the irradiation light 005, the width of the culturing container 002 (the width from the side close to the light emission unit 004 to the side far from the light emission unit 004), the transmittance of the irradiation light 005 estimated from the material of the culturing container 002, and the like, it is possible to calculate how much the amount of light has increased or decreased with respect to the amount of light of the referenced irradiation light 005 for each time point for each region of the culturing surface 001 and use the calculated result as the estimated irradiation light variation.

By adding the estimated irradiation light variation information to captured images, it is possible to remove unevenness in an image or temporal variation in images due to estimated variation in the irradiation light 005 and generate an image that would be obtained if the variation in the irradiation light 005 were not present.

The effect of suppressing variation in the amount of light due to absorption of light by the medium achieved by using the irradiation light 005 as described above becomes more notable as the image capturing range becomes wider, that is, the difference in optical length in the image capturing region becomes larger. A more significant effect is resulted when a generally used dish having a width of 30 mm or the culturing container 002 having a culturing surface area greater than this dish is used. That is, when the image capturing unit captures an image of a region of 30 mm×30 mm or greater, the cell observation system of the present embodiment achieves a more notable effect.

### Third Embodiment

The third embodiment of the present disclosure is a cell observation system having:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face;
an image capturing unit configured to capture an image of the culturing surface; and
an information processing unit,
wherein the information processing unit performs
an image information acquisition step of acquiring image information on a captured image from the image capturing unit,
a characteristic calculation step of calculating a characteristic of a medium in the culturing container from the image information, and
a characteristic output step of outputting the characteristic of the medium in the culturing container.

### Configuration of Cell Observation System

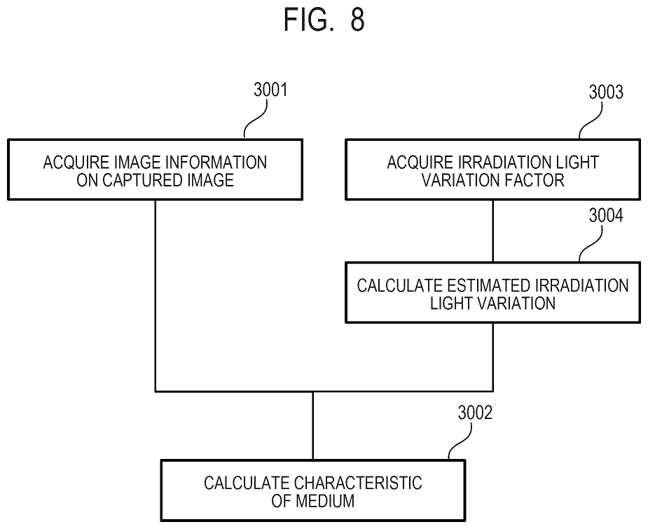

The cell observation system of the second embodiment illustrated in FIG. 5 can be referred for the configuration of the cell observation system of the present embodiment. As illustrated in FIG. 8, the information processing unit 201 acquires image information on images captured by the image capturing unit 006 (image information acquisition step 3001) and then calculates the characteristics of a medium in the culturing container from the image information (characteristic calculation step 3002).

Furthermore, the information processing unit may acquire an irradiation light variation factor (irradiation light variation factor acquisition step 3003), calculate an estimated irradiation light variation based on the irradiation light variation factor (estimated irradiation light variation calculation step 3004), and calculate the characteristics of the medium from the image information and the estimated irradiation light variation.

### Spectrum Characteristic of Irradiation Light

In the present embodiment, to calculate the characteristics of the medium, it is preferable that variation in the amount of light of the irradiation light 005 due to absorption of light by the medium in the culturing container 002 be obtained in a magnified manner, and in particular, it is preferable that the variation due to pH of the medium be large. It is therefore preferable for the irradiation light 005 to have spectrum characteristics corresponding to a high absorptivity due to the medium in the culturing container 002.

It is preferable for the irradiation light 005 to have the maximum intensity in a wavelength range of λ_{2A}−25 [nm] to λ_{2A}+25 [nm] or a wavelength range of λ_{2B}−25 [nm] to λ_{2B}+25 [nm] in the spectroscopic spectrum thereof, where the maximum absorption wavelength of the medium when the medium is acidic is λ_{2A} [nm] and the maximum absorption wavelength of the medium when the medium is alkaline is λ_{2B} [nm]. When the pH indicator of the medium is phenol red, it is preferable for the irradiation light 005 to have the maximum intensity in a wavelength range of 400 nm to 450 nm or a wavelength range of 525 nm to 575 nm in the spectroscopic spectrum thereof. When the medium contains a pH indicator other than phenol red, it is preferable to employ the irradiation light 005 having spectrum characteristics corresponding to the range described above in accordance with the characteristics of each pH indicator.

### Characteristic Calculation Step, Characteristic Output Step

The image information acquisition step 3001 is the same as that of the second embodiment. The characteristic calculation step 3002 will be described below. The characteristic calculation step is a step of calculating the characteristics of a medium in a culturing container. The characteristics of a medium refers to the characteristics of the medium in the culturing container 002 and may include the liquid amount of the medium, the pH of the medium, the elapsed time from start of culturing, the number of cultured cells, the state of proliferation of cultured cells, or the like, and more preferably may be the pH of the medium, the number of cultured cells, and the state of proliferation of cultured cells. The information processing unit 201 calculates these characteristics based on the image information on the captured image.

For example, it is assumed that light having the wavelength of λ_{2A} [nm] is used as the irradiation light 005 for irradiation, where the maximum absorption wavelength when the medium is acidic is λ_{2A} [nm] and the maximum absorption wavelength when the medium is alkaline is λ_{2B} [nm]. Since it is found that the irradiation light 005 is absorbed by the medium when the irradiation light 005 travels across the width of the culturing container 002 if the difference in brightness of images between a region close to the light emission unit 004 and a region far from the light emission unit 004 is higher than a certain level, characteristics that the medium has absorption at λ_{2A} [nm], that is, the medium is acidic can be calculated.

In the present embodiment, the information processing unit may perform the irradiation light variation factor acquisition step and the estimated irradiation light variation calculation step in addition to the characteristic calculation step and the characteristic output step. However, the irradiation light variation factor acquisition step and the estimated irradiation light variation calculation step are not necessarily required to be performed. The irradiation light variation factor is as described in the second embodiment. However, since the present embodiment calculates the characteristics of the medium, it is preferable that the irradiation light variation factors do not include those related to the characteristics of the medium to be calculated in the present embodiment.

Preferable examples of those to be calculated as the characteristics of the medium may be the pH of the medium, the number of cultured cells, and the state of proliferation of cultured cells. In contrast, the irradiation light variation factors may include the type of the light source of the light emission unit 004, the intensity, the spectroscopic spectrum, the irradiation distribution of irradiation light, or the like as the information on irradiation light; and the type, the material, the size, the capacity, the shape, the manufacturer, the model, or the like as the information on a culturing container. When the information on a medium or the like in a culturing container is used as the irradiation light variation factor, the information corresponds to the type of pigments contained in the medium, the elapsed time from start of culturing, the type of cultured cells, culturing conditions, or the like that can be easily input by the user.

Information on the correspondence between the captured image information and the characteristics of the medium may be stored in advance in the information processing unit 201, and the characteristics of the medium can be calculated from the captured image information. In this process, irradiation light variation factors may be further added, information on the correspondence between the captured image information, the irradiation light variation factors, and the characteristics of the medium may be stored in advance, and based thereon, the characteristics of the medium may be calculated from the captured image information and the irradiation light variation factor.

Alternatively, a reasoner may be provided, and the reasoner may be used to infer the characteristics of the medium from the captured image information. For example, the reasoner can use a learning model obtained by using training data in which image information is used as input data and characteristics of the medium are used as output data. Furthermore, in this example, in addition to the captured image information, a learning model obtained by using training data in which irradiation light variation factors are used as input data and characteristics of the medium are used as output data may be used.

### Characteristic Output Step

The calculated characteristics are output to a medium such as paper via, for example, a monitor screen, a screen of a tablet terminal or a smartphone, a writable optical drive, or a printer so that the user may recognize the calculated characteristics. By viewing the characteristics of the medium displayed on the characteristic output unit, the user of the cell observation system is able to confirm that the unevenness in an image or temporal variation in images is due to progress of culturing and is not due to a failure of the cell observation system itself. Further, it is possible to recognize that the state of the medium has changed and obtain information used for determining a timing to perform the next culturing step such as exchange of the culture medium.

Note that the feature of irradiating the side face with light according to the present system is significantly advantageous in the above calculation of medium characteristics, which enables a long optical path to be obtained even with a small amount of the medium and realizes improvement of accuracy in the calculation of medium characteristics.

If the characteristics of a medium (for example, pH of a culture medium) can be calculated, an irradiation light amount distribution in the culturing surface can be calculated. The image processing unit can correct unevenness of the amount of light by calculating such an irradiation light amount distribution and processing image information acquired by the image capturing unit 006. Further, it is also possible to add characteristic information on the medium to the image information, and this is effective in terms of management of images. For example, it is possible to write pH of the medium to an image or write pH information to a header part or a file name.

With respect to the variation in the amount of light due to absorption of light by a medium, the wider the image capturing range is, that is, the larger the difference in the optical path in the image capturing region is, the more notably the variation in the amount of light due to characteristics is obtained. That is, a more significant effect is resulted when a generally used dish having a width of 30 mm or the culturing container 002 having a culturing surface area greater than this dish is used. That is, when the image capturing unit captures an image of a region of 30 mm×30 mm or greater, the cell observation system of the present disclosure achieves a more notable effect.

### Program and Medium

The present disclosure includes a non-transitory storage medium storing the program in a computer readable form in each embodiment of the cell observation system described above.

Although the present invention has been described above with reference to the embodiments, the present invention is not limited to the embodiments described above. The invention that is changed from the present invention within the scope not contrary to the spirit of the present invention and the invention that is equivalent to the present invention are also included in the present invention. Further, the embodiments described above can be combined with each other as appropriate within the scope not contrary to the spirit of the present invention.

### Other Embodiments

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-166289, filed Sep. 27, 2023, which is hereby incorporated by reference herein in its entirety.

What is claimed is:

1. A cell observation system comprising:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face; and
an image capturing unit configured to capture an image of the culturing surface,
wherein the cell observation system has at least any one of features of
(a) controlling variation in an amount of light of the irradiation light, the culturing surface being irradiated with the irradiation light, and the variation being due to absorption of light by a medium in the culturing container, and
(b) correcting an effect caused by variation in an amount of light of the irradiation light, the culturing surface being irradiated with the irradiation light, and the variation being due to absorption of light by a medium in the culturing container.

2. The cell observation system according to claim 1,
wherein the cell observation system has the feature of (a), and
wherein intensities of the irradiation light at a wavelength λ_{2A} [nm] and a wavelength λ_{2B} [nm] in a spectroscopic spectrum of the irradiation light are less than or equal to 50% of the intensity at a wavelength λ₁ [nm], where
the intensity is the maximum at the wavelength λ₁ [nm] in the spectroscopic spectrum of the irradiation light,
absorption is the maximum at the wavelength λ_{2A} [nm] in an absorption spectrum of the medium when the medium is acidic, and
absorption is the maximum at the wavelength λ_{2B} [nm] in an absorption spectrum of the medium when the medium is alkaline.

3. The cell observation system according to claim 1,
wherein the cell observation system has the feature of (a), and
wherein the intensity of the irradiation light is the maximum in a wavelength range from 450 nm to 500 nm or a wavelength range of 600 nm or higher in the spectroscopic spectrum of the irradiation light.

4. The cell observation system according to claim 1,
wherein the cell observation system has the feature of (a), and
wherein the intensity of the irradiation light at a wavelength of 430 nm and 560 nm is less than or equal to 50% of the maximum intensity in the spectroscopic spectrum of the irradiation light.

5. The cell observation system according to claim 1,
wherein the cell observation system has the feature of (b) and further comprises an information processing unit,
wherein the information processing unit performs
an image information acquisition step of acquiring image information on an image captured by the image capturing unit,
an irradiation light variation factor acquisition step of acquiring an irradiation light variation factor,
an estimated irradiation light variation calculation step of calculating an estimated irradiation light variation based on the irradiation light variation factor, and
an image processing step of generating a processed image based on the image information and the estimated irradiation light variation.

6. The cell observation system according to claim 5, wherein the irradiation light variation factor is acquired based on at least any one selected from a group consisting of information on irradiation light, information on a culturing container, and information on a medium in a culturing container.

7. The cell observation system according to claim 6, wherein the information on the medium includes information on pH of the medium.

8. The cell observation system according to claim 6, wherein in the irradiation light variation factor acquisition step, the information on the medium is acquired by using the image information on a plurality of different regions on the culturing surface.

9. A cell observation system comprising:
a placement part for a culturing container to be placed, the culturing container having a culturing surface on which cells are culturable and having an optically transparent side face forming a peripheral wall of the culturing surface;
a light emission unit configured to irradiate the culturing surface with irradiation light from the side face;
an image capturing unit configured to capture an image of the culturing surface; and
an information processing unit,
wherein the information processing unit performs
an image information acquisition step of acquiring image information on a captured image from the image capturing unit,
a characteristic calculation step of calculating a characteristic of a medium in the culturing container from the image information, and
a characteristic output step of outputting the characteristic of the medium in the culturing container.

10. The cell observation system according to claim 9, wherein the information processing unit further performs
an irradiation light variation factor acquisition step of acquiring an irradiation light variation factor, and
an estimated irradiation light variation calculation step of calculating an estimated irradiation light variation based on the irradiation light variation factor.

11. The cell observation system according to claim 9, wherein the irradiation light has the maximum intensity in a wavelength range of λ_{2A}−25 [nm] to λ_{2A}+25 [nm] or a wavelength range of λ_{2B}−25 [nm] to λ_{2B}+25 [nm], where
the maximum absorption wavelength of the medium when the medium is acidic is λ_{2A} [nm], and
the maximum absorption wavelength of the medium when the medium is alkaline is λ_{2B} [nm].

12. The cell observation system according to claim 9, wherein the irradiation light has the maximum intensity in a wavelength range from 400 nm to 450 nm or a wavelength range from 525 nm to 575 nm in a spectroscopic spectrum of the irradiation light.

13. The cell observation system according to claim 9, wherein in the characteristic calculation step, the characteristic of the medium is calculated by using the image information on a plurality of different regions on the culturing surface.

14. The cell observation system according to claim 9, wherein the characteristic of the medium is pH.

15. The cell observation system according to claim 1, wherein the image capturing unit captures an image of a region of 30 mm×30 mm or larger.

16. A non-transitory storage medium storing a program that causes the information processing unit to perform the image information acquisition step, the irradiation light variation factor acquisition step, the estimated irradiation light variation calculation step, and the image processing step in the cell observation system according to claim 5.

17. A non-transitory storage medium storing a program that causes the information processing unit to perform the image information acquisition step, the characteristic calculation step, and the characteristic output step in the cell observation system according to claim 9.

18. A non-transitory storage medium storing a program that causes the information processing unit to perform the image information acquisition step, the irradiation light variation factor acquisition step, the estimated irradiation light variation calculation step, the characteristic calculation step, and the characteristic output step in the cell observation system according to claim 10.
